(19) United States
(12) Patent Application Publication
(10) Pub. No.: US 2015/0216788 A1
(43) Pub. Date: Aug. 6, 2015

(54) COMPOSITION FOR EXTRACTING INKS FROM SKIN

(71) Applicant: GEORGE D. PETITO, BETHLEHEM, PA (US)

(72) Inventor: GEORGE D. PETITO, BETHLEHEM, PA (US)

(21) Appl. No.: 14/521,444

(22) Filed: Oct. 22, 2014

Related U.S. Application Data
(60) Provisional application No. 61/935,131, filed on Feb. 3, 2014.

(51) Int. Cl. A61K 8/81; A61K 8/65; A61Q 1/14; A61K 8/73

(52) U.S. Cl. CPC A61K 8/8152; A61K 8/731; A61K 8/65; A61Q 1/145

(57) ABSTRACT

The composition for extracting inks from skin may be applied to the skin to remove ink, such as tattoo ink, from the dermis and/or subdermal layers of the skin. The composition may include effective amounts of collagen, carboxymethyl cellulose (CMC), and a carbomer. The composition may be applied to the skin after laser treatment of the skin.

### CROSS-REFERENCE TO RELATED APPLICATION

This application claims the benefit of U.S. Provisional Patent Application Serial No. 61/935,131, filed Feb. 3, 2014.

### BACKGROUND OF THE INVENTION

1. Field of the Invention

The present invention relates to removal of tattoos, and particularly to a composition for extracting inks from skin.

2. Description of the Related Art

Tattoo removal using traditional methods has disadvantages, such as incomplete pigment removal, non-selective tissue destruction, and unsatisfactory cosmetic results, such as atrophic or hypertrophic scarring. Traditional methods of tattoo removal have included abrasion with salt (salabrasion), cryosurgery, dermabrasion, electrocoagulation, and the use of an infrared coagulator. All of these procedures are associated with significant scarring, and the result after tattoo removal can appear worse than the tattoo alone. Conventional surgical methods have been used to treat tattoos. However, their use is limited to the removal of small tattoos. Surgical removal of large tattoos usually yields unacceptable results.

The use of lasers to remove tattoos began in the early 1990s. Lasers work by producing short pulses of intense light that pass harmlessly through the top layers of the skin to be selectively absorbed by the tattoo pigment. This laser energy causes the tattoo pigment to fragment into smaller particles that are then removed by the body's immune system. Researchers have determined which wavelengths of light to use and how to deliver the output of the laser effectively to remove tattoo ink. The laser does not affect normal skin pigment.

While laser removal of tattoos is increasingly becoming the most popular way to achieve tattoo removal, many patients experience ink retention following the tattoo removal treatment. In other words, even after the requisite number of laser treatments for tattoo removal has been completed, tattoo pigment often remains visible on the patients' skin.

Thus, a composition to improve tattoo ink extraction after laser surgery is desired.

### SUMMARY OF THE INVENTION

The composition for extracting inks from skin may be applied to the skin to remove ink, such as tattoo ink, from the dermis and/or subdermal layers of the skin. The composition may include effective amounts of collagen, carboxymethyl cellulose (CMC), and a carbomer. The composition may be applied to the skin after laser treatment of the skin.

These and other features of the present invention will become readily apparent upon further review of the following specification.

### DETAILED DESCRIPTION OF THE PREFERRED EMBODIMENTS

The composition for extracting inks from skin may be applied to the skin to remove ink, such as tattoo ink, from the dermis and/or subdermal layers of the skin. The composition may include effective amounts of collagen, carboxymethyl cellulose (CMC), and a carbomer.

The collagen may be native collagen (naturally occurring collagen) and/or hydrolyzed collagen. Native collagen typically has a molecular weight within the range of 100 to 300,000 Daltons. Hydrolyzed collagen is defined as a collagen hydrolysate polypeptide having a molecular weight lower than native collagen, and is derived by hydrolysis. Hydrolyzed collagen is commercially available in powdered form or as an aqueous solution. Commercial preparation is typically accomplished by one of four methods: (1) alkaline hydrolysis; (2) enzymatic hydrolysis; (3) acid hydrolysis; and (4) synthetically, by fermentation. Any of these methods can be used to derive the hydrolyzed collagen from either a bovine (bone and skin preferred), porcine, fish, avian, or a synthetic source.

Collagen may be combined with CMC (carboxymethyl cellulose), carbomer, and/or other similar polymers to facilitate ink removal. The other similar polymers may include, for example, polydextrose, xantham gum, guar gum, sodium alginate, carrageenan, hydroxypropyl cellulose (HPC), hydroxypropyl methylcellulose (IIPMC), methylcellulose, polyvinylpyrrolidone (PVP), maltodextrin, polyvinyl alcohol, polyethylene glycol (PEG), polyethylene oxide, and hydroxyethyl cellulose (HEC). An example of a preferred carbomer for use in the composition is a polymer having high molecular weight acrylic acid chains, usually crosslinked, e.g., carbomers commercially available from the Lubrizol Corp. under the trade name CARBOPOL.

The composition may be used to remove tattoo ink from the skin. The composition is particularly effective when applied to the skin after laser treatment for tattoo removal. In many cases, laser procedures for tattoo removal alone are not sufficient to remove all tattoo ink from the skin. The composition may be applied to the skin after the laser procedure in order to remove any remaining tattoo ink from the skin. When the skin is treated with the composition after the laser procedure, tattoo ink may be removed more efficiently than with laser treatment alone.

Laser treatment of the skin prior to application of the composition may facilitate penetration of the composition into the skin. The composition may be used after ablative or non-ablative laser treatment. Ablative laser treatment is generally more invasive than non-ablative laser treatment. Ablative laser treatment produces very small holes in the dermis, and facilitates chemical penetration through all layers of the skin. As non-ablative laser treatment is less invasive than ablative laser treatment, a lesser degree of penetration into the skin is facilitated thereby.

The amounts of the ingredients of the composition described above may vary, depending upon the type of laser treatment performed. For example, CMC is preferably a primary ingredient of the composition when the intended use of the composition is application after ablative laser treatment. In particular, when the composition includes greater amounts of CMC than collagen, better results may be achieved for applications after ablative laser treatment. When the composition is intended for use after non-ablative laser treatment, however, the primary ingredient of the composition is preferably collagen. In particular, when the composition includes greater amounts of collagen than CMC, better results may be achieved for applications after non-ablative laser treatment.

Preferably, for applications after ablative laser treatment, the composition may include from about 80% by weight to about 95% by weight CMC. For example, the composition may include from about 82% by weight to about 93% by weight, or from about 85% by weight to about 90% by weight of CMC. The composition may include an amount of from about 5% by weight to about 15% by weight collagen. For example, collagen may be present in an amount of from about 5% by weight to about 9% or about 10% by weight to about 15% based on the total weight of the composition. The composition may include from about 10% by weight to about 20% by weight carbomer. For example, the carbomer may be present in an amount of from about 12% by weight to about 17% by weight based on the total weight of the composition. Other amounts below and above these ranges may be used.

Preferably, for applications after non-ablative laser treatment, the composition may include from about 80% by weight to about 95% by weight collagen based on the total weight of the composition. For example, the composition may include from about 82% by weight to about 93% by weight, or from about 85% by weight to about 90% by weight, of collagen based on the total weight of the composition. The composition may include from about 10% by weight to about 20% by weight CMC based on the total weight of the composition. For example, CMC may be present in an amount of from about 12% to about 18% , or about 15% to about 20%, based on the total weight of the composition. The composition may include from about 5% by weight to about 15% by weight carbomer based on the total weight of the composition. For example, the carbomer may be present in an amount of from about 7% by weight to about 10% by weight based on the total weight of the composition. Other amounts below and above these ranges may be used.

The composition may include one or more absorption quality intermediates and/or one or more antibacterial compounds. A suitable absorption quality intermediate may be talc, for example. Suitable antibacterial compounds may be, for example, chlorhexidine, quaternium ammonium compounds, bacitracin zinc, chlortetracycline hydrochloride, neomycin sulfate, tetracycline hydrochloride, or polymyxin B.

The composition may include at least one auxiliary ingredient. The auxiliary ingredient may be platelet-shaped powders, metallic stearates, starch, china clay, tea tree powder, or polytetrafluoroethylene. The one or more auxiliary ingredients may be present in an amount of from about 0.1% by weight to about 15% by weight based on the total weight of the composition.

The composition may be administered topically. The composition may take any physical form suitable for topical administration. For example, the composition may be formed as a powder, paste, or film. The composition may be incorporated in a dressing bandage, i.e., reservoir island dressing, or a topically applied patch. The composition may be applied as part of a primary dressing and covered with a thin film wound dressing (e.g., polyurethane or acrylic dressing) or gauze, for occlusion purposes. Preferably, the composition is applied over the treated skin tissue in this manner immediately after laser treatment. The composition may be applied to the treated skin at least daily for at least the first three days after laser treatment.

Application of the composition facilitates removal of some or all remaining tattoo ink after laser treatment, as well as absorption of debris and exudate (fluid). The composition may also accelerate healing, prevent bacteriological infection of the treated skin, as well as minimize the possibility of scarring. Use of the composition does not result in maceration of the surrounding skin.

### Example 1

### Composition A

The composition according to the present teachings was synthesized in powder form. The composition (Composition A) included about 85% by weight CMC, about 10% by weight carbopol, and about 5% by weight collagen. The ingredients were mixed using standard methods for achieving uniformity and dispersion. Composition A was tested on numerous individuals for ink absorption, healing, and scarring levels after ablative laser treatment for tattoo removal. All tattoo ink colors were tested. Application of Composition A after ablative laser treatment was found to remove 75-85% of tattoo ink remaining after the laser treatment.

### Example 2

### Composition B

The composition according to the present teachings was synthesized in powder form. The composition (Composition B) included about 85% CMC, about 5% by weight carbopol, about 5% by weight collagen, and about 5% by weight of one or more antibacterial compounds. The antibacterial compounds tested included chlorhexidine, quaternium ammonium compounds, bacitracin zinc, chlortetracycline hydrochloride, neomycin sulfate, tetracycline hydrochloride, and polymyxin B. The ingredients were mixed using standard methods for achieving uniformity and dispersion. Composition B was tested on numerous individuals for ink absorption, healing, and scarring levels after ablative laser treatment for tattoo removal. All tattoo ink colors were tested. Application of Composition B after ablative laser treatment was found to remove slightly less tattoo ink than Composition A. Composition B, however, was associated with faster healing time and less pain than Composition A.

It is to be understood that the present invention is not limited to the embodiments described above, but encompasses any and all embodiments within the scope of the following claims.

I claim:

1. A composition, comprising:
carboxymethyl cellulose;
collagen selected from the group consisting of native collagen and hydrolyzed collagen; and
a carbomer.

2. The composition according to claim 1, wherein:
carboxymethyl cellulose is present in an amount of about 80% by weight to about 95% by weight;
collagen is present in an amount of about 5% by weight to about 15% by weight; and
carbomer is present in an amount of about 10% by weight to about 20% by weight.

3. The composition according to claim 2, further comprising at least one absorption quality intermediate or at least one antibacterial compound.

4. The composition according to claim 3, wherein the at least one absorption quality intermediate comprises talc.

5. The composition according to claim 3, wherein the at least one antibacterial compound comprises at least one of chlorhexidine, quaternium ammonium compound, bacitracin zinc, chlortetracycline hydrochloride, neomycin sulfate, tetracycline hydrochloride, and polymyxin B.

6. The composition according to claim 2, further comprising at least one absorption quality intermediate and at least one antibacterial compound.

7. The composition according to claim 6, wherein the at least one absorption quality intermediate comprises talc.

8. The composition according to claim 6, wherein the at least one antibacterial compound comprises at least one of chlorhexidine, quaternium ammonium compound, bacitracin zinc, chlortetracycline hydrochloride, neomycin sulfate, tetracycline hydrochloride, and polymyxin B.

9. The composition according to claim 1, wherein:
carboxymethyl cellulose is present in an amount of about 10% by weight to about 20% by weight;
collagen is present in an amount of about 80% by weight to about 95% by weight; and
carbomer is present in an amount of about 5% by weight to about 15% by weight.

10. The composition according to claim 9, further comprising at least one absorption quality intermediate and/or at least one antibacterial compound.

11. The composition according to claim 10, wherein the at least one absorption quality intermediate comprises talc.

12. The composition according to claim 10, wherein the at least one antibacterial compound comprises at least one of chlorhexidine, quaternium ammonium compound, bacitracin zinc, chlortetracycline hydrochloride, neomycin sulfate, tetracycline hydrochloride, and polymyxin B.

13. A composition, consisting essentially of:
carboxymethyl cellulose;
collagen selected from the group consisting of native collagen and hydrolyzed collagen; and
a carbomer.

14. The composition according to claim 13, wherein:
carboxymethyl cellulose is present in an amount of about 80% by weight to about 95% by weight;
collagen is present in an amount of about 5% by weight to about 15% by weight; and
carbomer is present in an amount of about 10% by weight to about 20% by weight.

15. The composition according to claim 13, wherein:
carboxymethyl cellulose is present in an amount of about 10% by weight to about 20% by weight;
collagen is present in an amount of about 80% by weight to about 95% by weight; and
carbomer is present in an amount of about 5% by weight to about 15% by weight.

16. A method of removing a tattoo, comprising the step of applying a composition to a skin portion bearing the tattoo, the composition comprising collagen, carboxymethyl cellulose, and a carbomer.

17. The method according to claim 16, wherein:
carboxymethyl cellulose is present in an amount of about 80% by weight to about 95% by weight of the composition;
collagen is present in an amount of about 5% by weight to about 15% by weight of the composition; and
carbomer is present in an amount of about 10% by weight to about 20% by weight of the composition.

18. The method according to claim 16, wherein:
carboxymethyl cellulose is present in an amount of about 10% by weight to about 20% by weight of the composition;
collagen is present in an amount of about 80% by weight to about 95% by weight of the composition; and
carbomer is present in an amount of about 5% by weight to about 15% by weight of the composition.
